(19) United States
(12) Patent Application Publication
(10) Pub. No.: US 2025/0230427 A1
(43) Pub. Date: Jul. 17, 2025

(54) SYSTEM AND METHOD FOR INCREASING THE TRANSDUCTION CAPABILITY AND REPROGRAMMING EFFICIENCY IN TARGETING CELLS AND TISSUES

(71) Applicant: National Yang Ming Chiao Tung University, Hsinchu City (TW)

(72) Inventors: Guang-Yuh CHIOU, Hsinchu City (TW); Chian-Shiu CHIEN, Taipei City (TW)

(73) Assignee: National Yang Ming Chiao Tung University, Hsinchu City (TW)

(21) Appl. No.: 18/413,886

(22) Filed: Jan. 16, 2024

(51) Int. Cl. C12N 13/00; C12M 1/32; C12N 5/00

(52) U.S. Cl. CPC C12N 13/00; C12M 23/12; C12N 5/0012

(57) ABSTRACT

The present invention provides a system for enhancing efficiency of gene delivery and/or reprogramming of target cells or tissues, comprising a magnet field-generating device that is assembled in a 12-well culture plate and magnet components to generate a magnet field, and magnet beads, which are used to label targeting cells or tissues, or which are incorporated into agarose gel. Also provided is the method using the system,

### FIELD OF THE INVENTION

The present invention pertains to a system and method for increasing the transduction capability and reprogramming efficiency in targeting cells and tissues.

### BACKGROUND OF THE INVENTION

Gene therapy is an advanced intervention technology that can replace the defective genes or restore the deficient genes in the host cells. Gene delivery can be achieved predominantly via using either viral or non-viral vectors. After the removal of pathogenicity and self-replication capabilities, viral vectors can be used as ideal tool for gene delivery to meet the demands of gene therapy or laboratory experiments. Viral vectors can be further divided into integrating vectors and non-integrating. Integrating viral vectors, such as retroviruses and lentiviruses, can integrate their genetic information into the patient DNA. Non-integrating viral vectors, such as adenoviruses, adeno-associated viruses (AAV), and herpes simplex viruses (HSV), retain the episomal DNA in the cytoplasm without integration. Episomal vectors do not replicate themselves in the patient's DNA and disappear within a short time.

Mesenchymal stem cells (MSCs) hold promising cell-based therapeutic application in degenerative diseases and tissue repair. Currently, most of the majorities of the MSCs are collected from adipose tissues or cord blood biopsies. By this method, MSCs are collected from various sources and the safety and cell operation procedures are exposed to high uncertainties in the regards of GTP-competent cell products. MSCs-derived from induced pluripotent stem cells (iPSCs) are newly established protocols for generating high quality MSCs and the batch-to-batch variations are dramatically reduced. By this protocol, mass production of GTP-competent MSCs to meet biomedical needs of huge amount of MSCs with high standard of safety and cell quality becomes possible and the MSCs quality control and production protocols can be standardized and quantitatively measurable in the laboratories fitting GTP standards. In this regard, two major hurdles of the iPSCs-MSCs protocols are: efficiencies of establishing iPSCs and differentiation and quality control of MSCs derived from iPSCs.

The conventional methods for establishing iPSCs require transductions of foreign genetic materials. For example, plasmids which expresses iPSCs reprogramming genes consisting of OCT4, SOX3, Klf4 and c-Myc. Various reprogramming protocols had established and the efficiency of reprogramming is validated and tested. Our team previously had successfully established a iPSCs reprogramming method by replacing c-Myc with non-oncogenic PARP1 gene. The replacement of c-Myc with PARP1 generates high genome stability and the pluripotency are comparable to original iPSCs induction protocols established by Takahashi K et al. All of these methods require delivery of exogenous stemness genes into somatic cells or peripheral blood mononuclear cells (PBMCs) to induced cell reprogramming into iPSCs. The choices of gene delivery vehicles are including retrovirus, episomal vector, or recently developed Sendai virus (SeV) or direct delivery of expression vectors expressing required reprogramming stemness genes. These methods are facing safety concern of genome integrations except iPSCs reprogrammed from SeV, which the chances of genome integration is minimal. Recently, advancing in RNA technology and nanoparticles lift the bar in biomedical applications. With the biodegradable and non-genome integrating properties of RNA/nanoparticle combination, it significantly reduces the safety concern in establishing iPSCs. Besides, the RNAs and nanoparticles can be fully synthesized and produced in GMP-competent laboratories or factories. The production sources are controllable, tunable as compared with conventional laboratories-prepared expression vectors or virus soups for transfections.

Mesenchymal stem cells (MSCs) are critical cell sources for cell-based biomedical applications including tissue repair and differed degeneration. MSCs hold promising cell-based therapeutic application in degenerative diseases and tissue repair. Currently, most of the majorities of the MSCs are collected from adipose tissues or cord blood biopsies. The MSCs are lineage committed stem cells with limited cell differentiation capacities. The safety of MSCs in biomedical applications is high due to the limited pluripotency of MSCs. MSCs hold promising cell-based therapeutic application in degenerative diseases and tissue repair. Currently, most of the majorities of the MSCs are collected from adipose tissues or cord blood biopsies. By this method, MSCs are collected from various sources and the safety and cell operation procedures are exposed to high uncertainties in the regards of GTP-competent cell products. However, the sources of MSCs are the major safety concern of MSCs in biomedical applications. Majority of the MSCs are collected from biopsies by flowcytometry based cell sorting and separation methods. Pathogen-free MSCs are extremely difficult to obtain albeit pathogen detections form MSCs collected from various biopsies are conducted. Still, GTP competent MSCs production processes are major challenges in using MSCs as cell-based therapeutic reagents. Production of pathogen-free iPSCs are more feasible than MSCs collected from various uncontrollable sources. Generations of iPSCs and differentiations of iPSCs to MSCs can be conducted in GTP competent laboratories or cell producing facilitated with controllable operations and parameters. MSCs prepared from these approaches have promising potentials in personalized/autologous or allogeneic cell transplantation and cell-based therapeutics. However, the purity of MSCs-derived from iPSCs is a major concern of production quality of MSCs derived from iPSCs.

MSCs hold promising cell-based therapeutic application in degenerative diseases and tissue repair. Currently, most of the majorities of the MSCs are collected from adipose tissues or cord blood biopsies. By this method, MSCs are collected from various sources and the safety and cell operation procedures are exposed to high uncertainties in the regards of GTP-competent cell products. MSCs-derived from induced pluripotent stem cells (iPSCs) are newly established protocols for generating high quality MSCs and the batch-to-batch variations are dramatically reduced. By this protocol, mass production of GTP-competent MSCs to meet biomedical needs of huge amount of MSCs with high standard of safety and cell quality becomes possible and the MSCs quality control and production protocols can be standardized and quantitatively measurable in the laboratories fitting GTP standards. In this regard, two major hurdles of the iPSCs-MSCs protocols were established, but the efficiencies of the production of iPSCs, differentiation and quality control of MSCs derived from iPSCs still need to be enhanced.

The conventional methods for establishing iPSCs require transductions of foreign genetic materials. For example, plasmids which expresses iPSCs reprogramming genes consisting of OCT4, SOX3, Klf4 and c-Myc. Various reprogramming protocols had established and the efficiency of reprogramming is validated and tested. Our team previously had successfully established a iPSCs reprogramming method by replacing c-Myc with non-oncogenic PARP1 gene. The replacement of c-Myc with PARP1 generates high genome stability and the pluripotency are comparable to original iPSCs induction protocols established by Takahashi K et al. All of these methods require delivery of exogenous stemness genes into somatic cells or peripheral blood mononuclear cells (PBMCs) to induced cell reprogramming into iPSCs. The choices of gene delivery vehicles are including retrovirus, episomal vector, or recently developed Sendai virus (SeV) or direct delivery of expression vectors expressing required reprogramming stemness genes. These methods are facing safety concern of genome integrations except iPSCs reprogrammed from SeV, which the chances of genome integration is minimal. Recently, advancing in RNA technology and nanoparticles lift the bar in biomedical applications. With the biodegradable and non-genome integrating properties of RNA/nanoparticle combination, it significantly reduces the safety concern in establishing iPSCs. Besides, the RNAs and nanoparticles can be fully synthesized and produced in GMP-competent laboratories or factories. The production sources are controllable, tunable as compared with conventional laboratories-prepared expression vectors or virus soups for transfections.

The transduction of genetic materials from any given vector into the host cells relies on the intensive exposure of vectors to the target cells or tissues. However, especially for those suspension culture or tissue slice, these host cells/tissues cannot effectively sink in the culture media, therefore reducing the probably of the vector-to-cell contact and suppressing the transduction efficiency of exogenous genes. For example, the unstable gene transduction can be observed in the reprogramming of suspension somatic cells. In addition, tissue slices that tended to be floating failed to be infected by viruses in the culture. Based upon these drawbacks in conventional setup for vector-mediated gene transduction or viral infections, an efficiency method that can enhance the contact between given vectors and host cells/tissues should be required.

Accordingly, it is desired to develop a method and device to effectively and efficiently produce iPSCs, and increase the transduction capability and reprogramming efficacies in targeting cells and tissues.

### BRIEF SUMMARY OF THE INVENTION

The present invention provides a system and method by combing a novel device design with novel biomaterials to accelerate the production and quality assurance of iPSCs, wherein the quality and easy-of-operations of isolating MSCs derived from iPSCs can be achieved from the novel devices and utilizations of the newly formulated biomaterials.

In one aspect, the invention provides a system for enhancing efficiency of gene delivery and/or reprogramming of target cells or tissues, comprising a magnet field-generating device, which is assembled in a 12-well culture plate with magnet components including ring-shaped magnet, a magnet holder and a passive aligner to generate a magnet field, and magnet beads, which are used to label targeting cells or tissues, or which are incorporated into agarose gel.

In one example of the invention, the magnet field-generating device comprises a ring-shaped magnet; a magnet holder; a culture plate; and a passive aligner; wherein the ring-shaped magnet and the magnet holder are assembled, and then assembled in the culture plate.

According to the invention, the magnet adhesion of the target cells or tissues under the magnet filed increases the contact probability of cell-to-cell, cell-to-virus or antibody-to-target in the bottom of the culture plates during the process of viral infection or gene transduction. The magnet field-generating system and method are able to enhance the transduction efficiency of the exogenous genes carried by any given bead-labeled vector.

In one example of the invention, a non-integrating self-amplifying RNA (saRNA) is prepared and encapsuled with nanoparticles based on modified iron oxide/PEI formulations, in combinations with customized 3D-printing prepared magnetic microfluidic device. The magnetic device is modified with specialized hydrophobic surface modification for enhancing transfection efficiencies of transductions and iPSCs generations. All these improvements together in iPSCs production significantly increase the successful rate in iPSCs productions and due to high gene delivery efficiency, the amounts of cells required in iPSCs production is significantly reduced and therefore decreasing the technical challenges in establishing iPSCs. The time- and cost-requirements for generating personalized and customized made iPSCs are cost feasible by these methods.

According to the invention, a magnet field-generating device is constructed by using 3D-printing technologies, which can be assembled in a 12-well culture plate and constitutively generate the magnet field. Next, the target cells/tissues were labeled by magnet beads or incorporated into a magnet bead-containing agarose gel. By these modifications, the magnet adhesion of the target cells or tissues under the magnet filed will increased the contact probability of cell-to-virus in the bottom of culture plates during the process of viral infection and gene transduction. Accordingly, the device and the 3D-printing-based magnet field-generating method enhance the transduction efficiency of exogenous genes carried by any given bead-labeled vector.

According to the invention, the system can be used for differentiating iPSCs into target cells. This magnetic device can be utilized in selecting specific cells derived from iPSCs. For example, induced mesenchymal stem cells (iMSCs) may be used. The differentiated cells are isolated with iron oxide/protein A/antibody complex and iMSCs with suitable expressions of surface markers are attached to the bottom of cell plates and the desired cells are isolated. The method provides a straightforward isolations of target cells with the whole procedures of less than 10 minutes and the yield of iMSCs with high purities. The whole procedures of using modified iron oxide materials and 3D printed devices for the processes of iPSCs producing to isolations of cells derived from iPSCs.

In one further aspect, the present invention provides an easy-to-operate, scalable cell separation and production method based on magnetic facilitated antibodies for purifying MSCs with specific MSCs surface marker sets. The method provides an affinity based MSCs separation and purification method with the customizable fluidic device by 3D-printing to maximize the in-well MSCs purifications and cost reduction. By the method, the drawbacks in conventional setup for vector-mediated gene transduction or viral infection have been improved, and the contact between given vectors and host cells/tissues is enhanced to improve the efficacy.

In a further aspect, the present invention provides a magnet field-generating device, and target cells/tissues labeled by magnet beads or incorporated into a magnet bead-containing agarose gel. For example, the magnet field-generating device may be constructed by using 3D-printing technologies, which can be assembled in a culture plate and constitutively generate the magnet field. By using the device of the invention, the transduction capability and reprogramming efficiency in targeting cells and tissues are increased.

According to the present invention, the magnet adhesion of the target cells or tissues under the magnet field will increase the contact probability of cell-to-virus in the bottom of culture plates during the process of viral infection and gene transduction. The device and the novel 3D-printing-based magnet field-generating method according to the invention are able to enhance the transduction efficiency of exogenous genes carried by any given bead-labeled vector.

According to the invention, the method has the following features:
1. A 3D-printing-based magnet field-generating device is constructed, which can be assembled under a 12-well culture plate and provide a constitutive and strong magnet field to the culture.
2. The magnet field will promote the landing of bead-labeled culture cells which facilitate the viral infection of cells and enhance the transfection efficiency and resultant expression of exogenous gene.
3. This magnet field-generating device can be effectively applied in gene delivery/transfection, the generation of induced pluripotent stem cells, and viral infection study.

The features and advantages of the present invention will be apparent to those skilled in the art. While numerous changes may be made by those skilled in the art, such changes are within the scope of this invention.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

The drawings include:

FIG. 1 provides a diagram showing the concept to the invention, characterized by an innovative 3D-printer-based magnet field-generating device to enhace the gene delivery efficiency (e.g. CAR-T therapy) or reprogramming efficacy of iPSC generation.

FIG. 2 shows the components of the innovative 3D-printing-based magnet field-generating device according to the invention, and the assembly of the advic. The left column of FIG. 2 showing the components of the device, comprising (a) ring-shaped magnets, (b) magnet holders, and (c) passive aligner. The right column of FIG. 2 shows the process for assembling the device, comprising (1) getting the three components (i.e., ring-shape magnet, the magnet holder, and the passive aligner) at the top-down position before assembly; (2) fitting the ring-shape magnet into the magnet holder and then combining the holder with the passive aligner.

FIG. 3 shows the magnet adhesion under the magnet field generated by the ring-shaped magnet, comprising cells labeled with magnetic beads, and the gravity and the magnet field guide the magnetic beads to land on the bottom of the well. The transfection and reprogramming efficiencies determined by counting eGFP-positive colonies and positive colonies of AP staining, wherein PBMC were transfected with retrovirus factors without or with magnetic devices. FIG. 3(A) shows the procedure of the combination of the device and a culture plate, which comprises the steps of (A) placing the 12-well culture plate below the device, (B) gently combining them together; wherein the 12 ring-shaped magnets were aligned to the commercial 12-well culture plate passively after complete assembly; and in the assembly of the device and the commercial 12-well dish is shown in (C), each aligned ring-shaped magnet providing a symmetrical magnetic field for each well (D). FIG. 3(B) shows the process of the magnet adhesion of the target cells or tissues under the magnet filed to increases the contact probability. FIG. 3(C) shows the comparison in transfection efficacy between the blank (None), the processes with and without the magnetic device according to the invention. FIG. 3(D) shows the comparison in reprogramming efficacy between the blank (None), the processes with and without the magnetic device according to the invention.

FIG. 4 shows the enhanced PBMC reprogramming by iron oxide/PEI/saRNAs; wherein PBMCs were transfected with formulated iron oxide/PEI/saRNA nanoparticles with magnetic devices. FIG. 4(A) shows the scheme of iron oxide/PEI/saRNAs mediated transfections. In brief, formulated iron oxides were coated with PEI and then co-incubated with saRNAs containing open reading frames of Yamanaka factor, SOX2, OCT4, c-Myc, and Klf4. FIG. 4(B) shows the transfection abilities were validated with iron oxide/PEI/eGFP formulations. FIG. 4(C) shows that the formulated iron oxide/PEI/saRNAs nanoparticles were incubated with PBMCs and the transfection efficiencies were direct compared with conventional transfection methods including liposome-, retrovirus-, and iron oxide/PEIs. FIG. 4(D) shows that 5 individual colonies were isolated and the expressions of selective stemness genes including Oct4, Sox2, Nanog, Rex1 were analyzed by semi-quantitative PCRs. These results shown that formulation of iron oxide/PEI was capable to conduct gene deliveries in PBMCs and induce reprogramming of PBMCs as evidenced by inductions of Nanog expression.

FIG. 5 shows that the magnet field-generating device increased the efficiency of iPSC reprogramming from PBMCs. FIG. 5(A) shows that the endogenous Oct4 expression was higher in reprogramming cells transduced with the magnet field-generating device than that without the device. FIG. 5(B) shows that the stemness gene Nanog presented a similar pattern as using the magnet field-generating device. FIG. 5(C) shows that three days after the delivery of OSKM (Oct4, Nanog, Sox2, and c-Myc) genes, the expression of Oct4 and Nanog was increased in reprogramming cells transduced with the magnetic devices group. FIG. 5(D) shows that the relative reprogramming efficiency was estimated by alkaline phosphatase staining and showed higher efficiency in reprogramming cells with the magnet field-generating devices than that without the device.

FIG. 6 provides the results of the iron oxide/PEI/saRNAs mediated iPSCs reprogramming method. Formulated iron oxides are coated with PEI at 1 mg/mL for 16 h. PEI-coated iron oxides are isolated by magnetic precipitations. Self-amplifying RNAs are constructed by inserting genes-of-interests (GOIs) down stream of nsp1-4 genes which are essential genes for RNA self-replications. The transfection of iron oxide/PEI/GOI were confirmed by transfection hTERT-RPE cells. The iPSCs cell reprogramming was confirmed by transfecting human cells with iron oxide/PEI/saRNAs (reprogramming genes) and the stemness gene expressions and iPSCs alkaline phosphatase activities were tested and confirmed.

FIG. 7 shows that the formulations of iron oxides can be further extended to cell sorting; wherein iron oxides coated with protein A/G and conjugated with antibodies specific for surface marker of MSCs are a replacement of FACS. Mixtures of MSCs derived from iPSCs can be isolated and purified by formulations of iron oxides/protein A/G/antibodies. All these procedures of MSCs isolations and purifications were in-a-well performance. Combing with customized magnetic devices, the whole procedures can be finished within 15 minutes. The purities of isolated MSCs derived from iPSCs are further validated with flowcytometry analysis as shown below. These results showed the purities of MSCs isolated by combos of iron oxides/protein A/G/antibodies and magnetic devices are comparable to conventional FACS.

FIG. 8 shows that the SARS-COV2 infection efficiency was increased in the organotypic culture assembled with the magnetic field-generating device. FIG. 8(A) shows the schematic illustrations of virus infection (SARS-COV2 or pseudovirus) in the organotypic culture system. FIG. 8(B) provides the images of the H & E stain, which show the 3D structure of the human lung slice culture on the plates assembled with the magnetic field-generating device. FIG. 8(C) shows that human lung slice (bottom) expressed SARS-COV-2 receptor ACE2 (Green) and nuclei (PI, Red). FIG. 8(D) shows that the magnet field-generating device enhanced SARS-COV2 infection of the organotypic culture. FIG. 8(E) shows that the magnet field-generating device enhanced GFP-expressing lentiviral infection of iPSCs.

### DETAILED DESCRIPTION OF THE INVENTION

The foregoing summary, as well as the following detailed description of the invention, will be better understood when read in conjunction with the appended drawings. For the purpose of illustrating the invention, there are shown in the drawings embodiments which are presently preferred.

The present invention provides an easy-to-operate, scalable cell separation and production method based on magnetic facilitated antibodies for purifying MSCs with specific MSCs surface marker sets. Therefore, the invention provides an affinity based MSCs separation and purification method with a customizable fluidic device by 3D-printing to maximize the in-well MSCs purifications and cost reduction. By the method, the drawbacks in conventional setup for vector-mediated gene transduction or viral infection have been improved, and the contact between given vectors and host cells/tissues is enhanced to improve the efficacy.

By these modifications, the magnet adhesion of the target cells or tissues under the magnet filed will increased the contact probability of cell-to-virus in the bottom of culture plates during the process of viral infection and gene transduction. Plausibly, our device and the novel 3D-printing-based magnet field-generating method is able to enhance the transduction efficiency of exogenous genes carried by any given bead-labeled vector.

In one example of the invention, a non-integrating self-amplifying RNA (saRNA) is encapsuled with nanoparticles based on modified iron oxide/PEI formulations, in combinations with customized 3D-printing prepared magnetic microfluidic device. The magnetic device is modified with specialized hydrophobic surface modification for enhancing transfection efficiencies of transductions and iPSCs generations. All these improvements together in iPSCs production significantly increase the successful rate in iPSCs productions and due to high gene delivery efficiency, the amounts of cells required in iPSCs production is significantly reduced and therefore decreasing the technical challenges in establishing iPSCs. The time-and cost-requirements for generating personalized and customized made iPSCs are cost feasible by these methods.

Based upon these backgrounds, a magnet field-generating device is constructed by using 3D-printing technologies, which can be assembled in a 12-well culture plate and constitutively generate the magnet field. Next, the target cells/tissues were labeled by magnet beads or incorporated into a magnet bead-containing agarose gel. By these modifications, the magnet adhesion of the target cells or tissues under the magnet filed will increased the contact probability of cell-to-virus in the bottom of culture plates during the process of viral infection and gene transduction. Plausibly, the device and the novel 3D-printing-based magnet field-generating method are able to enhance the transduction efficiency of exogenous genes carried by any given bead-labeled vector.

According to the invention, the system can be used for differentiating iPSCs into target cells. This magnetic device can be utilized in selecting specific cells derived from iPSCs. For example, induced mesenchymal stem cells (iMSCs) may be used. The differentiated cells are isolated with iron oxide/protein A/antibody complex and iMSCs with suitable expressions of surface markers are attached to the bottom of cell plates and the desired cells are isolated. The method provides a straightforward isolations of target cells with the whole procedures of less than 10 minutes and the yield of iMSCs with high purities. The whole procedures of using modified iron oxide materials and 3D printed devices for the processes of iPSCs producing to isolations of cells derived from iPSCs.

Accordingly, the present invention provides an easy-to-operate, scalable cell separation and production method based on magnetic facilitated antibodies for purifying MSCs with specific MSCs surface marker sets. Therefore, the invention provides an affinity based MSCs separation and purification method with a customizable fluidic device by 3D-printing to maximize the in-well MSCs purifications and cost reduction. By the method, the drawbacks in conventional setup for vector-mediated gene transduction or viral infection have been improved, and the contact between given vectors and host cells/tissues is enhanced to improve the efficacy.

Based upon these drawbacks in conventional setup for vector-mediated gene transduction or viral infection studies, an efficiency method that can enhance the contact between given vectors and host cells/tissues should be required.

Based upon these backgrounds, we used 3D-printing technologies to construct a magnet field-generating device that can be assembled in a commercial 12-well culture plates and constitutively generate the magnet field. Next, the target cells/tissues were labeled by magnet beads or incorporated into a magnet bead-containing agarose gel. By these modifications, the magnet adhesion of the target cells or tissues under the magnet filed will increased the contact probability of cell-to-virus in the bottom of culture plates during the process of viral infection and gene transduction. Plausibly, our device and the novel 3D-printing-based magnet field-generating method is able to enhance the transduction efficiency of exogenous genes carried by any given bead-labeled vector.

The invention provides a non-integrating self-amplifying RNA (saRNA) encapsuled with nanoparticles based on modified iron oxide/PEI formulations, furthermore, in combinations with the customized 3D-printing prepared magnetic microfluidic device. The magnetic device was modified with specialized hydrophobic surface modification to enhance transfection efficiencies of transductions and iPSCs generations. All these improvements in iPSCs production significantly increase the successful rate in iPSCs productions due to high gene delivery efficiency. The amounts of cells required in iPSCs production is significantly reduced to decrease the technical challenges in establishing iPSCs. The time-and cost-requirements for generating personalized and customized made iPSCs are cost feasible by these methods.

The following applications of differentiating iPSCs into target cells according to the invention are provided. This magnetic device can be utilized in selecting specific cells derived from iPSCs. For example induced mesenchymal stem cells (iMSCs). The differentiated cells were isolated with iron oxide/protein A/antibody complex and iMSCs with suitable expressions of surface markers were attached to the bottom of cell plates and the desired cells were isolated. This method provided a straightforward isolations of target cells with the whole procedures of less than 10 minutes and the yield of iMSCs with high purities. The whole procedures of using modified iron oxide materials and 3D printed devices for the processes of iPSCs producing to isolations of cells derived from iPSCs are summarized as below:

According to the invention, 3D-printing technologies are used to construct a magnet field-generating device that can be assembled in a commercial 12-well culture plate and constitutively generate the magnet field. Next, the target cells/tissues were labeled by magnet beads or incorporated into a magnet bead-containing agarose gel. By these modifications, the magnet adhesion of the target cells or tissues under the magnet filed will increased the contact probability of cell-to-virus in the bottom of culture plates during the process of viral infection and gene transduction. Plausibly, our device and the novel 3D-printing-based magnet field-generating method is able to enhance the transduction efficiency of exogenous genes carried by any given bead-labeled vector.

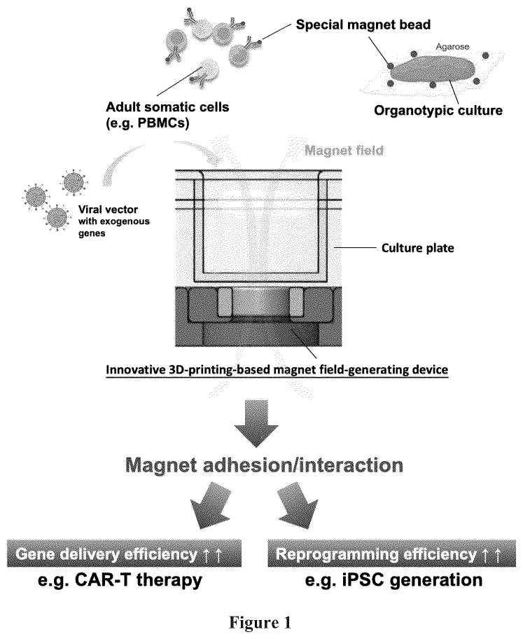

The concept of the present invention is illustrated in FIG. 1, including:
1. Considering the sinking nature of viral vectors, we aimed to construct a 3D-printing-based magnet field-generating device that can promote the sinking of target cells to the bottom of culture plates, leading to the increased probability of the physical contact between viral vectors and target cells.
2. To enable the magnet adhesion/interaction between the magnet field and target cells, we designed to label the target cells with antibody-conjugated special magnet beads, allowing them to be attract to the bottom of culture plates in the presence of magnet field.
3. For organotypic culture that tends to float in the media, we attempted to encapsulate the target tissue (organotypic culture) in the low-melting agarose gel containing special magnet beads within it.
4. The 3D-printing-based magnet field-generating device is able to be assembled for most of any given commercial 12-well culture plates. After the assembly, the device is able to constitutive generate strong magnet field.
5. The intensive contact/interaction between viral vectors and target cells/tissues are supposed to enhance the viral infection and gene transduction efficiency.
6. This novel 3D-printing-based magnet field-generating device that can enhance gene transduction efficiency can be utilized in the biomedical application including iPSC generation and CAR-T therapy.

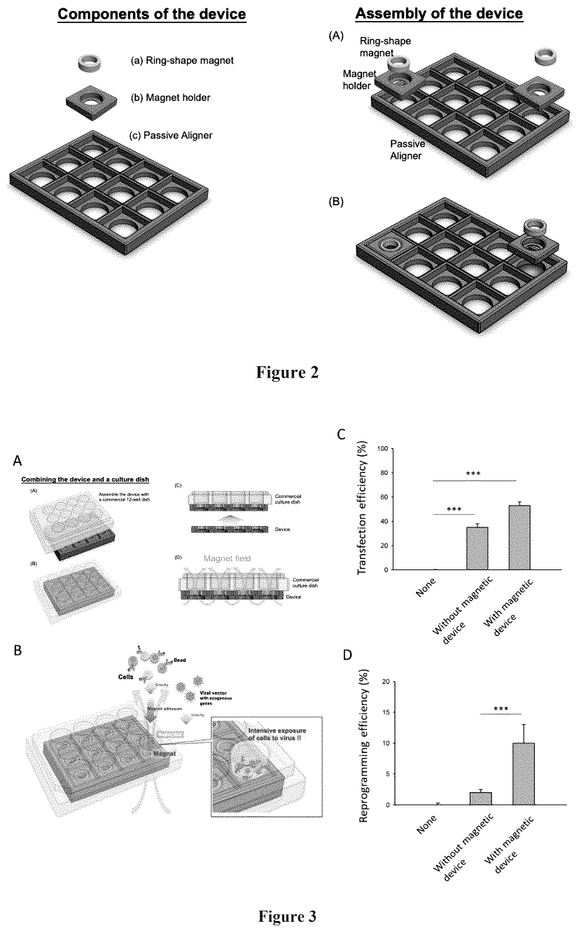

The setup and assembly of the innovative 3D-printing-based magnet field-generating device are shown in FIG. 2. As shown in the left column of FIG. 2, three components of the innovative 3D-printing-based magnet field-generating device, (a) ring-shaped magnet, (b) magnet holder, and (c) passive aligner. In the right column showing the assembly of the device comprises (A) the top-down position of three components before assembly: ring-shape magnet, the magnet holder, and the passive aligner; and (B) fit the ring-shape magnet into the magnet holder and then combine the holder with the passive aligner.

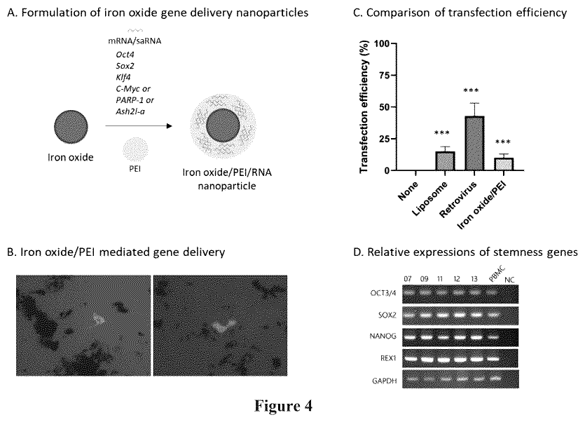

As shown in FIG. 3, the commercial 12-well culture plate is placed below the device and they are gently combined together. The 12 ring-shaped magnets were aligned to the commercial 12-well culture dish passively after complete assembly. FIG. 3(C) and FIG. 3(D) shows the comparison in transfection efficacy, and reprogramming efficacy between the blank (None), the processes with and without the magnetic device according to the invention.

As shown in FIG. 3(B), the components and the assembly process according to the invention include (1) representative ring-shaped magnets and magnet holders, (2) A commercial 12-well culture dish, an unassembled ring-shaped magnet, an unassembled magnet holder, and a passive aligner with two representative assembled magnet holders with ring-shaped magnets; (3) assemble the ring-shaped magnet into the magnet holder; (4) assemble the magnet holder with a ring-shaped magnet into the passive aligner; (5) assemble the passive aligner into the commercial 12-well culture dish.

In conclusion, the present invention provides the following features:
(a) A 3D-printing-based magnet field-generating device was contructed that can be assembled under any given commercial 12-well culture plate and provide a constitutive and strong magnet field to the culture.
(b) The magnet field will promote the landing of bead-labeled culture cells which facilitate the viral infection of cells and enhance the transfection efficiency and resultant expression of exogenous gene.
(c) This magnet field-generating device can be effectively applied in gene delivery/transfection study, the generation of induced pluripotent stem cells, and viral infectiond.
(d) The 3D-printing device with magnetic field can facilitate iPSCs production by delivering RNAs/nanoparticles into PBMCs.
(e) The magnetic device can separate and purify MSCs or any cells with specific surface markers with magnetic material modified antibodies in well.
(f) A formulation of mRNA/nanoparticles with a magnetic device was provided to improve reprogramming efficiencies and safety of reprogramming somatic cells into induced pluripotent stem cells.
(g) These methods present nanoparticles containing iron oxide, PEI and RNAs as gene delivery vehicles for wide range of somatic cells including adhesive and suspension cells.
(h) The complete device design including a magnetic field generating device, 3D-printing generated cell culture supporting dishes or microfluidic devices and formulated nanoparticles.
(i) These methods presented by our team is suitable for gene delivery into both adhesion and suspension cells for generating induced pluripotent stem cells.
(j) The compositions, including particle sizes of iron oxides, types and concentrations of PEIs, and contents of RNAs of the nanoparticles for gene delivery device (cell reprogramming device) is tailorable.
(k) The 3D-printing dishes and magnetic fields are tailorable in our device.
(l) These nanoparticles are able to conjugate with antibodies for magnetic field mediated antibody-based cell sorting by specific surface antigens presented by cells.
(m) These nanoparticles conjugated with antibodies are able to purify MSCs derived from human iPSCs but not only human iPSCs or human biopsy samples but not only human biopsies.

The present invention is further illustrated by the following examples, which are provided for the purpose of demonstration rather than limitation.

### EXAMPLES

### Example 1

### Retrovirus Transfection and Reprogramming Efficiencies was Enhanced by the Magnetic Device According to the Invention

The transfection and reprogramming edffection efficiencies were determined by counting eGFP-positive colonies and positive colonies of AP staining. PBMC were transfected with retrovirus factors without or with magnetic devices. The magnetic device enhanced retrovirus delivery were determined in FIG. 3(C). The reprogramming efficiencies of PBMC to iPSCs were determined by AP staining. These colonies were counted and quantitated as shown in FIG. 3(D). The regrogramming data showed significant among 2.5 folds increase in reprogramming effciencies with magnetic device.

### Example 2

### PBMC Reprogramming was Enhanced by Iron Oxide/PEI/saRNAs.

PBMCs were transfected with formulated iron oxide/PEI/saRNA nanoparticles with magnetic devices. The scheme of iron oxide/PEI/saRNAs mediated transfections were shown in FIG. 4(A). In brief, formulated iron oxides were coated with PEI and then co-incubated with saRNAs containing open reading frames of Yamanaka factor, SOX2, OCT4, c-Myc, and Klf4. The transfection abilities were validated with iron oxide/PEI/eGFP formulations as shown in FIG. 4(B). Then, the formulated iron oxide/PEI/saRNAs nanoparticles were incubated with PBMCs and the transfection efficiencies were direct compared with conventional transfection methods including liposome- , retrovirus- , and iron oxide/PEIs as shown in FIG. 4(C). 5 individual colonies were isolated and the expressions of selective stemness genes including Oct4, Sox2, Nanog, Rex1 were analyzed by semi-quantitative PCRs as shown in FIG. 4(D). These results shown that formulation of iron oxide/PEI was capable to conduct gene deliveries in PBMCs and induce reprogramming of PBMCs as evidenced by inductions of Nanog expression.

### Example 3

The Magnet Field-Generating Device Increased the Efficiency of iPSC Reprogramming

To estimate whether the magnetic device improved the efficiency of iPSCs from PBMCs. In the magnetic device group, PBMCs were collected and further labeled with the CD16-conjugated beads to promote PBMCs to land at the bottom of the plate under the magnet field. Inducible OSKM lentivirus was employed to reprogram PBMC into iPSCs. Endogenous stemness genes OCT4 and NANOG were evaluated by real-time PCR after the reprogramming of PBMCs transduced in the culture plates with or without the magnetic device. The expression of endogenous OCT4 was increased in cells with the magnetic device compared to those without the device. Another stemness marker NANOG showed a similar trend in cells transduced in the culture plates with the device. Next, western blot results showed consistent trends of the mRNA expression; OCT4 and NANOG were up-regulated after the reprogramming of PBMCs transduced in the culture plates with the device. Alkaline phosphatase staining indicated that the PBMCs with the magnetic device enhanced the efficiency of PBMC reprogramming. These results revealed that the magnetic device effectively increased the virus infection rate and induced the iPSC reprogramming from PBMCs.

As shown in FIG. 5, the magnet field-generating device increased the efficiency of iPSC reprogramming from PBMCs. As shown in FIG. 5(A), endogenous Oct4 expression was higher in reprogramming cells transduced with the magnet field-generating device than that without the device. As shown in FIG. 5(B), the stemness gene Nanog presented a similar pattern as using the magnet field-generating device. As shown in FIG. 5(C), three days after the delivery of OSKM (Oct4, Nanog, Sox2, and c-Myc) genes, the expression of Oct4 and Nanog was increased in reprogramming cells transduced with the magnetic devices group. As shown in FIG. 5(D), the relative reprogramming efficiency was estimated by alkaline phosphatase staining and showed higher efficiency in reprogramming cells with the magnet field-generating devices than that without the device.

### Example 4

### Reprogramming of Iron Oxide/PEI/saRNAs Mediated iPSCs

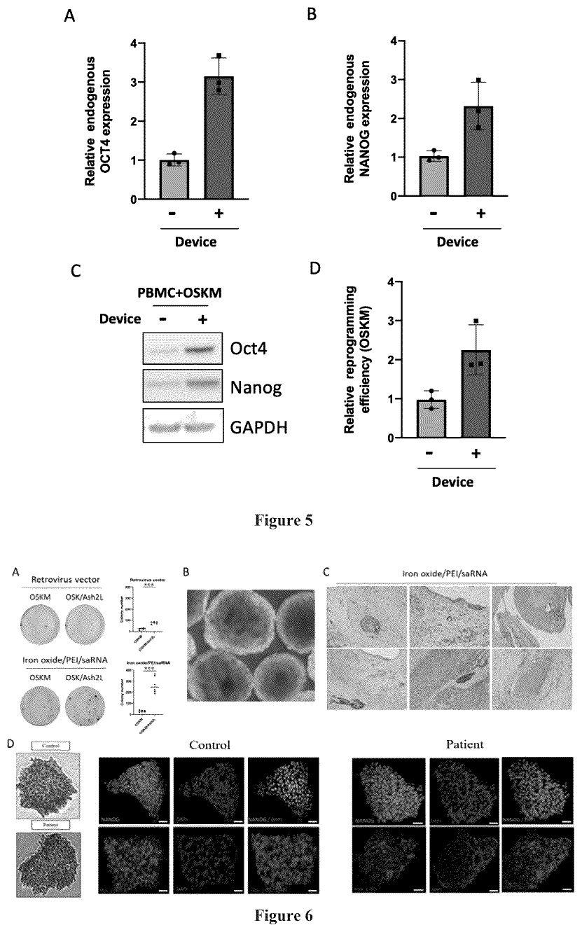

We prepared an iron oxide/PEI/saRNAs mediated iPSCs reprogramming method. Formulated iron oxides are coated with PEI at 1 mg/mL for 16 h. PEI-coated iron oxides are isolated by magnetic precipitations. Self-amplifying RNAs are constructed by inserting genes-of-interests (GOIs) down stream of nsp1-4 genes which are essential genes for RNA self-replications. The transfection of iron oxide/PEI/GOI were confirmed by transfection hTERT-RPE cells. The iPSCs cell reprogramming was confirmed by transfecting human cells with iron oxide/PEI/saRNAs (reprogramming genes) and the stemness gene expressions and iPSCs alkaline phosphatase activities were tested and confirmed. The results are shown in FIG. 6.

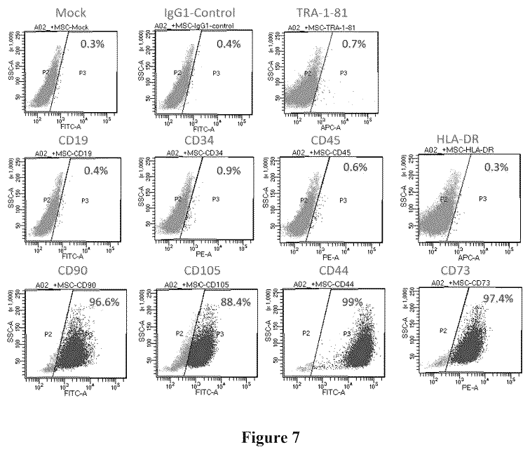

The formulations of iron oxides can be further extended to cell sorting. Iron oxides coated with protein A/G and conjugated with antibodies specific for surface marker of MSCs are a replacement of FACS. Mixtures of MSCs derived from iPSCs can be isolated and purified by formulations of iron oxides/protein A/G/antibodies. All these procedures of MSCs isolations and purifications were in-a-well performance. Combing with customized magnetic devices, the whole procedures can be finished within 15 minutes. The purities of isolated MSCs derived from iPSCs are further validated with flowcytometry analysis as shown in FIG. 6 and FIG. 7. These results showed the purities of MSCs isolated by combos of iron oxides/protein A/G/antibodies and magnetic devices are comparable to conventional FACS.

### Example 5 Determination of the SARS-COV2 Infection Rate in Organotypic Culture with the Magnetic Device.

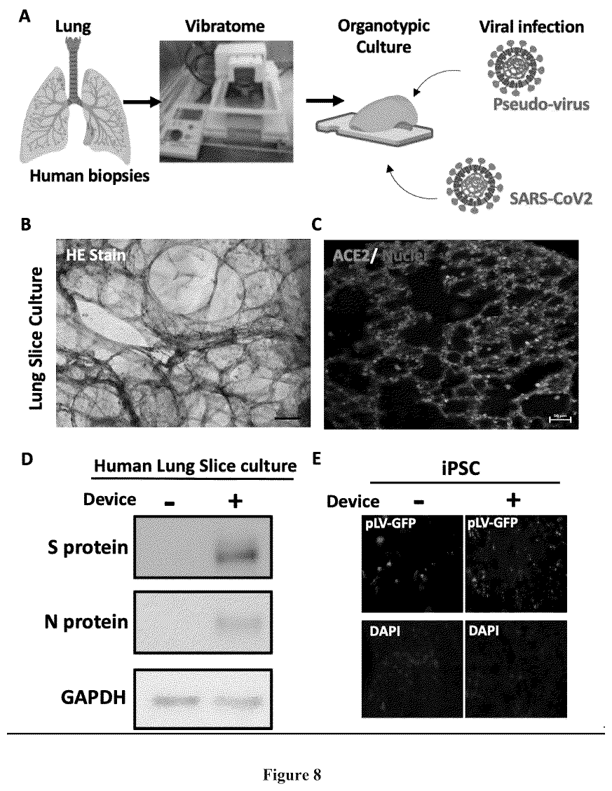

An in vitro platform for virus infection is critical for the screening of anti-viral medications. However, tissue slices that tended to float failed to be infected by viruses in the culture. To overcome this drawback of inability of tissue slices for viral infection, we examined whether the magnet field-generating device can promote viral infection in lung tissue slices. Human lung tissue was collected from patients who received lung cancer resection and was immediately sent to the laboratory for embedding in a low melting agarose gel. Tissue slices with a thickness of 200 μm were obtained through a vibrating microtome and further subjected to the infection SARS-COV-2 pseudovirus and virus. HE staining and immunostaining showed that the organotypic culture system maintained the structure and characteristics of lung tissue, and the expression of ACE2, the SARS-COV-2 entry receptor, was detectable (FIG. 8(B)). After the addition of SARS-COV-2 into the culture, Western blot results showed that the expression of S and N proteins were not detectable in the organotypic culture system infected in a culture plate without the magnet field-generating device. This could be due to the low exposure and contact of the slices to the viruses in the culture, leading to the low expression of viral proteins in the host cells. With the magnet field-generating device, the expression of S and N proteins were detectable in the lung slice culture, indicating that the magnet field-generating device promoted the successful infection of host cells by SARS-COV-2 (FIG. 8(C)). Lentivirus infection assay showed similar results; cells exposed to the green fluorescent protein (GFP)-expressing lentivirus in the presence of the magnetic device and field improved the efficiency of virus infection (FIG. 8(D)). All the data demonstrated that the magnet field-generating device induced the virus infection efficiency in both of the organotypic culture system and iPSCs.

In view of the results, the SARS-COV2 infection efficiency was increased in the organotypic culture assembled with the magnetic field-generating device.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

What is claimed is:

1. A system for enhancing efficiency of gene delivery and/or reprogramming of target cells or tissues, comprising a magnet field-generating device that is assembled in a 12-well culture plate and magnet components to generate a magnet field, and magnet beads, which are used to label targeting cells or tissues, or which are incorporated into agarose gel

2. The system of claim 1, in which the magnet field-generating device comprises a ring-shaped magnet, a magnet holder, a culture plate; and a passive aligner; wherein the ring-shaped magnet and the magnet holder are assembled, and then assembled in the culture plate.

3. The system of claim 1, in which the target cell or tissue is prepared by using a non-integrating self-amplifying RNA (saRNA) and encapsuling with a nanoparticle based on modified iron oxide/PEI formulation.

4. A method enhancing efficiency of gene delivery and/or reprogramming of target cells or tissues, using the system as set forth in claim 1.

5. The method of claim 4, in which the magnet field-generating device comprises a ring-shaped magnet, a magnet holder, a culture plate; and a passive aligner; wherein the ring-shaped magnet and the magnet holder are assembled, and then assembled in the culture plate.

6. The method of claim 5, in which the target cell or tissue is prepared by using a non-integrating self-amplifying RNA (saRNA) and encapsuling with a nanoparticle based on modified iron oxide/PEI formulation.
